(19) United States
(12) Patent Application Publication
(10) Pub. No.: US 2005/0038407 A1
(43) Pub. Date: Feb. 17, 2005

(54) Drug delivery warning system

(76) Inventor: James M. Sumka, Basking Ridge, NJ (US)

Correspondence Address: ERNEST D BUFF & ASSOCIATES, LLC, 245 SOUTH ST, MORRISTOWN, NJ, 07960, US

(21) Appl. No.: 10/638,909

(22) Filed: Aug. 11, 2003

(51) Int. Cl. A61M 31/00

(52) U.S. Cl. 604/500

(57) ABSTRACT

A drug delivery system has a drug delivery article for administering a medicament to a patient. The system is associated with a recognition article, which is correlated with an industry-recognized standard, for signaling a caregiver that precautions must be taken before administering the medicament to the patient. A high visibility, imperative is thereby provided, which warns against inadvertent administration of a pharmacological agent into the wrong site of a patient. The system preferably comprises a syringe bearing a red marking that signals the caregiver to avoid intrathecal injection of medicament therewithin. A signal triggered by the red marking may additionally warn that the contents of the drug delivery article are hazardous, and that caution must be undertaken to prevent the potentially fatal injection of an incorrect pharmaceutical solution into a patient.

### BACKGROUND OF THE INVENTION

1. Field of the Invention

The present invention relates to a drug delivery warning system; and more specifically to a warning system that comprises a drug delivery article and a recognition article operative to warn the user that the medicament within the drug delivery article should not be administered intrathecally.

2. Description of the Prior Art

Medication errors are an important cause of patient morbidity and mortality. One common cause of medication error involves administration of the wrong drug. In some cases this is the result of a caregiver grabbing the wrong syringe. It is common practice to simultaneously deliver multiple drugs. The ability to quickly and accurately identify which syringe is delivering which medicament is extremely important when various medicaments are being delivered simultaneously, particularly in emergency or surgical situations. The ability to distinguish various syringes even in the environment of an emergency response situation is very important. Syringe swaps, where a caregiver picks up the wrong syringe and administers the medicament into the wrong site on the patient, continue to occur even when the syringes are labeled.

It is extremely difficult if not impossible to distinguish one drug from another once the drugs have been withdrawn from their vials. In many cases the medicaments are administered using the same type and size of syringe. Errors, such as administration of an incorrect medication or an incorrect dose, will readily occur if the contents of the syringe cannot be positively identified from the point of time that the medicament is withdrawn from its vial up to the moment of its administration. Once the medication is withdrawn from its vial there is little opportunity to recheck its identity.

There is no universal method for distinguishing the contents of one syringe from another. Each medical practice adopts its own system for establishing and communicating the contents of a syringe. In some medical practices the medical professional preparing the injections assumes responsibility for devising his or her own system

It is well understood that efforts should be taken to decrease the occurrence of adverse drug events. Thus far a successful system for preventing administration of the wrong drug has not been discovered. Manufacturers of syringe-administered medications use several techniques to help medical professionals identify the contents of their vials. These techniques include color-coded labels and somewhat standard labeling arrangements that convey key information, including generic name, trade name, expiration date and lot numbers. However, once these medications are transferred from a vial into a syringe, the techniques used to identify the medication in that syringe provide few positive controls and are subject to failure.

Several other methods are commonly used to distinguish one injectable medication from another. Syringes have been placed on a medicine tray with an information card for each syringe. Stickers or permanent color indicia have been used to mark syringes. Color-coded labels have been used to identify syringes. However, these methods have been shown to be unreliable. The organization of medicine trays with syringes and their corresponding information cards can be disturbed when the tray is tipped or moved abruptly. Permanent markers on a syringe can be easily smudged by damp hands or altered by hands that have just used an alcohol prep to clean the top of a vial. Information stickers have to be separately prepared and may not be readily available when the medication is being drawn. An incorrect sticker may be used. Syringe swaps are not eliminated by the use of color coded labels. Thus, there remains a need for a system that would reliably prevent the administration of a drug into the wrong site of a patient.

One drug that has been linked to fatalities due to its accidental administration in the wrong location is vincristine. This vinca alkaloid is used extensively in chemotherapy because of its anti-tumor activity and lack of myelosyuppression at dosages commonly used in combination with other chemotherapeutic agents. Vincristine is safe when administered intravenously but potentially fatal when given intrathecally. In many instances, vincristine is administered with other medicaments on the same day. There have been several incidences where intrathecal administration of vincristine resulted in the death of a patient. There is a strong need in the art for an accurate, inexpensive system that would reliably warn an administrator of this medicament that the medicament should not be given intrathecally.

### SUMMARY OF THE INVENTION

The invention provides a drug delivery warning system that especially suited for use in the medical field to prevent the inadvertent, potentially fatal injection of a pharmaceutical solution into the wrong site of a patient. The system has a prominent recognition article for warning the caregiver that medicament within the syringe must not be administered intrathecally. It can also convey a warning that medicament within the drug delivery article must be administered with utmost caution; that administration of the medicament to the wrong body site may be extremely harmful to the patient. Further, the recognition article can convey a warning that that injection of the medicament is restricted solely to a preselected site of a patient.

Generally stated, the drug delivery warning system comprises a drug delivery article and a recognition article. The recognition article warns users that the medicament within the drug delivery article must not be administered intrathecally. Depending on the medicament, the recognition article may also warn users against administration of the medicament at other body sites that may be adversely affected thereby. The drug delivery article is preferably a syringe; and the recognition article comprises a red colored marking on the syringe.

This drug warning system is correlated with an industry recognized standard within a medical community, which conveys a warning that the contents of the drug delivery article are hazardous, and that caution must be undertaken to prevent the potentially fatal injection of an incorrect pharmaceutical solution into a patient.

The drug warning system is easy to implement by a medical community, owing to its relative simplicity and low operating cost. Due to its simplicity, the drug warning system is readily implemented by a medical community as an industry standard, providing clear recognition that medicament within the syringe must be administered with caution. A medical community that is cost-conscious would welcome an inexpensive system especially suited to help prevent syringe swaps—a problem frequently associated with the fatal intrathecal injection of vincristine into a patient.

### BRIEF DESCRIPTION OF THE DRAWINGS

The invention will be more fully understood and further advantages will become apparent when reference is had to the following detailed description of the preferred embodiment of the invention and the accompanying drawings, in which:

FIG. 1a is a perspective view of a syringe depicting the recognition article as a red strip running longitudinally down the syringe;

FIG. 1b is a perspective view of a Syringe depicting the recognition article as comprising a red colored plunger

FIG. 1c is a perspective view of the Syringe depicting the recognition article as comprising a red colored entire syringe;

FIG. 2 is a perspective partial cut-out view of the Syringe depicting the recognition article as comprising a red colored plunger;

FIG. 3a is a perspective view of the Syringe depicting the recognition article as comprising a red colored plunger and flange; and

FIG. 3b is a perspective view of the Syringe depicting the recognition article as comprising a red colored finger press.

### DETAILED DESCRIPTION OF THE INVENTION

As used herein, the term drug delivery article means a vehicle for administering a pharmacological agent to a patient including syringes, needles used in the intravenous delivery of pharmacologic agents and other such articles.

The term “industry standard” means an accepted standard within the medical establishment within a particular community. This community may be local (for example a particular hospital), regional, statewide, or nationwide.

The term “prominent” means easily visible upon a quick glance. The recognition article must be easily visible to the user to ensure that the user can quickly determine (i) that the medicament within a drug delivery article must not be given intrathecally, or (ii) that the medicament within the drug delivery article must be administered with the utmost caution to the patient in that administration of the medicament to the wrong body site may be exceedingly harmful to the patient.

The term “recognition article” means a prominently displayed marking on the syringe that correlates with an industry standard within a medical community denoting (i) that the medicament within the drug delivery article must not be administered intrathecally, or (ii) that the medicament within the drug delivery article must be administered with the utmost caution to the patient in that the administration of the medicament within the drug delivery article to the wrong body site may be fatal to the patient.

The present invention provides a drug delivery system aimed at preventing the potentially fatal administration of medicaments to a patient due to the administration of the medicament to the wrong body site. A prominent recognition article on the drug delivery system would warn users not to administer the medicament intrathecally or to take extra precautions before administering the drug. The recognition article can comprise a prominent marking, such as a colored part of the syringe or specifically colored labels. Red colored markings on the syringe are preferred; when used, such red colored markings denote “warning, use caution when administering the medicament within this syringe”. The recognition article is correlated with a recognized industry standard, which conveys a warning that (i) the contents of the drug delivery system are hazardous; and (ii) caution must be undertaken to prevent the potentially fatal injection of a medicament into the wrong site in a patient.

The recognition article could be a colored marking on the drug delivery article that comprises the entire drug delivery article, one or more components of the drug delivery article or even one section of one of the components of the drug delivery article. The recognition article can be layered onto the drug delivery article or embedded within the material making up the drug delivery article. The preferred colored marking would be red, a color that symbolizes “danger”. A medical community could easily adopt an industry standard that represents that a syringe bearing a red marking signifies that the medicament within the syringe should not be administered intrathecally.

The preferred drug delivery article would be a syringe. The syringe may be composed of thermoplastic resins, glass or any material that is commonly used in the production of syringes. The syringe would preferably comprise a barrel for containing a fluid medicament and a plunger. Index markings to allow the user to determine the amount of medicament within the syringe may be placed on either the plunger or barrel.

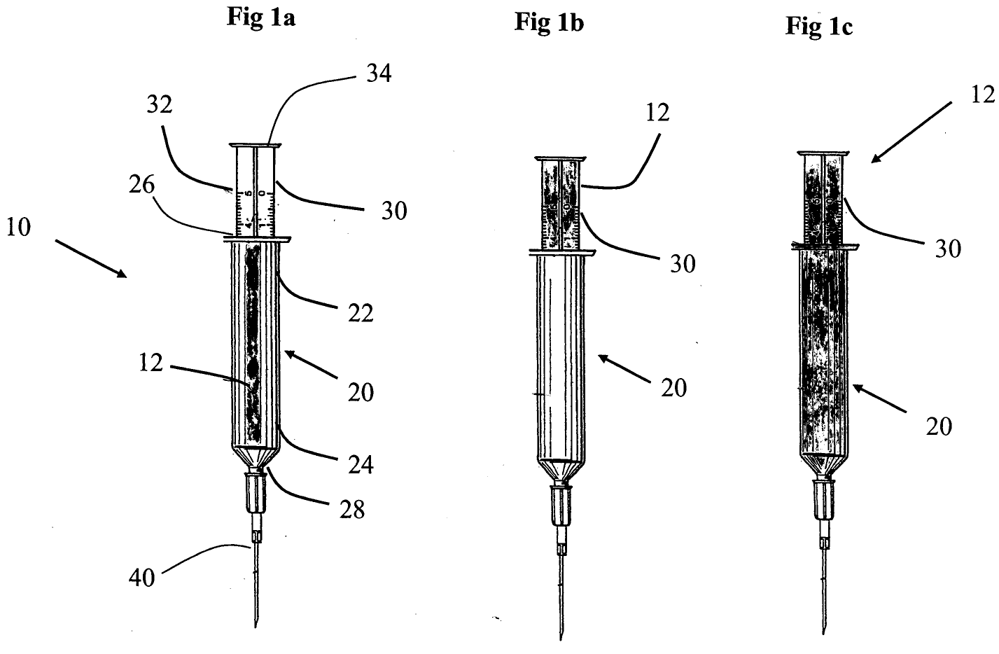

In One embodiment of the drug warning system is shown in FIG. 1a. In this embodiment the drug delivery article is a syringe 10. The syringe 10 comprises a barrel 20, having a proximal end 22 and a distal end 24 along a central longitudinal axis. The barrel 20 acts as a reservoir for fluid inside the syringe. The distal end of the barrel 24 has an aperture for permitting fluid exchange with the interior reservoir of the barrel. A tapered frusto-conical shaped tip 28 on the distal end of the barrel is adapted to accept a cannula or needle 40. The proximal end 22 of the barrel is open to receive a plunger 30. A flange 26 at the proximal end of the barrel facilitates handling and positioning the syringe barrel. The distal end of the plunger 38 fits into the proximal end of the barrel 22. The plunger 30 includes a finger press 34 on the proximal end of the plunger 36 and a seal member 39 at the distal end of the plunger 38. In this embodiment the plunger includes index markings 32. The recognition article comprises a red marking 12 running longitudinally down the barrel.

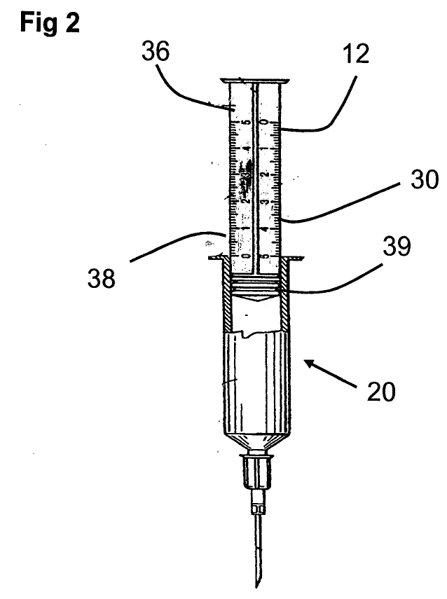

Another embodiment of the drug warning system is depicted in FIG. 1b and FIG. 2, in which the recognition article includes a red marking 12 on the plunger 30. In this embodiment the entire plunger is composed of the red marking 12. It will be understood by those skilled in the art that variations of this embodiment may be used, wherein only a portion of the plunger is composed of the red marking.

FIG. 1c depicts another embodiment of the syringe in which the entire syringe 10 is composed of the red marking 12.

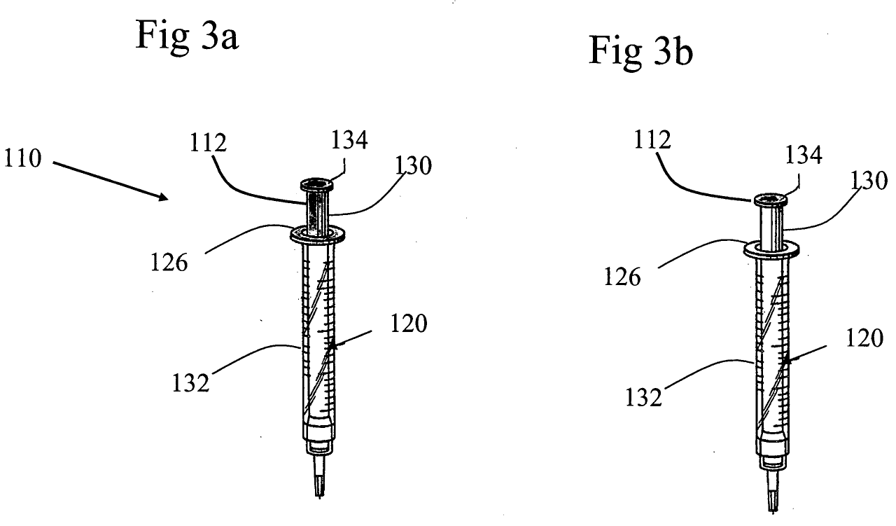

A syringe 110, in which the index markings 132 are on the barrel 120, is depicted by FIG. 3a and FIG. 3b. In the embodiment shown by FIG. 3a the entire plunger 130 and flange 126 are composed of the red marking 112. In FIG. 3b the finger press 134 is composed of the red marking 112.

The red marking may comprise a surface coating composed of a red material, which is placed on the syringe or a component part thereof; or it may comprise a red colored dye embedded into the material of which the syringe is composed or a component part thereof. It is important that the red marking be readily apparent to the user when the syringe is placed on a nearby surface, so that a user can distinguish the red marked syringe of the present invention with other syringes nearby, which are not similarly marked. Preferably, the red marking is applied so that index markings on the syringe are not concealed thereby. Likewise application of the red marking would preferably not be operative to prevent a user from observing the fluid level within the syringe barrel.

The index markings are typically applied to the syringe using an ink printing process. However there are numerous other ways to apply the index markings, including embossed application or molding of the index markings onto the syringe, which are thereafter printed or altered by color applications to effect high contrast volume index markings on the syringe. Such various marking means for application of index markings onto the syringe are contemplated as falling within the scope of the appended claims.

Having thus described the invention in rather full detail, it will be understood that such detail need not be strictly adhered to but that further changes and modifications may suggest themselves to one skilled in the art, all falling within the scope of the invention as defined by the subjoined claims.

1. A drug delivery system comprising:
a. a drug delivery article for administering a medicament to a patient; and
b. a recognition article correlated with an industry-recognized standard, for signaling a caregiver that precautions must be taken before administering said medicament to said patient.

2. A system as recited by claim 1 wherein said drug delivery article is a syringe comprising a barrel and a plunger, each of said barrel and said plunger having a distal end and a proximal end, and said distal end of said plunger being adapted to fit into the proximal end of said barrel.

3. A system as recited by claim 2 wherein said recognition article comprises an at least partially colored portion of said barrel.

4. A system as recited by claim 2 wherein said recognition article said partially colored portion is red.

5. A system as recited by claim 2 wherein said recognition article comprises an at least a partially colored portion of said plunger.

6. A system as recited by claim 2 wherein said recognition article comprises said least a partially colored portion is red.

7. A system as recited by claim 2 wherein said recognition article comprises an at least a partially colored barrel portion and a partially colored plunger portion.

8. A system as recited by claim 2 wherein said at least partially colored barrel and plunger portions are red.

9. A system as recited by claim 2 wherein said syringe further comprises a finger pad located at a proximal end of said plunger.

10. A system as recited by claim 7 wherein the recognition article comprises an least a partially colored portion of said finger pad.

11. A system as recited by claim 7 wherein said at least partially colored finger pad portion is red.

12. A method for injecting a fluid medicament, comprising the steps of:
i. placing a medicament within a drug delivery article associated with a recognition article, said recognition article being correlated with a recognized industry standard denoting that injection of said medicament is restricted solely to a preselected site of a patient;
ii. injecting said medicament from said drug delivery article into said preselected site of said patient.

13. A method as recited by claim 8, wherein the drug delivery article comprises a syringe having a barrel and a plunger, each of said barrel and said plunger have a distal end and a proximal end, and the distal end of said plunger is adapted to fit into the proximal end of said plunger.

14. A method as recited by claim 13, wherein said recognition article comprises an at least a partially colored barrel portion.

15. A method as recited by claim 13, wherein said at least partially colored barrel portion is red.

16. A method as recited by claim 13 wherein said at least a partially colored plunger portion is red.

17. A method as recited by claim 13 said recognition article comprises a red plunger.

18. A method as recited by claim 13 wherein said recognition article comprises at least partially colored barrel and plunger portions.

19. A system as recited by claim 13 wherein said partially colored barrel and plunger portions are red.

20. A method as recited by claim 13 wherein said syringe further comprises a finger pad located at the proximal end of said plunger, said finger pad having an at least partially colored portion.

21. A method as recited by claim 20 wherein said at least partially colored finger pad portion is red.

22. A method as recited by claim 20 wherein said recognition article comprises a red finger pad.
